(19) United States
(12) Patent Application Publication
(10) Pub. No.: US 2006/0172423 A1
(43) Pub. Date: Aug. 3, 2006

(54) New expression system from rhodococcus

(76) Inventors: Robert Van Der Geize, Veendam (NL); Gerda Hessels, Groningen (NL); Lubbert Dijkhuizen, Zuidlaren (NL); Peter Van Der Meijden, Oss (NL)

Correspondence Address: F Aaron Dubberly;Akzo Nobel Inc., Intellectual Property Department, 7 Livingstone Avenue, Dobbs Ferry, NY, 10522, US

(21) Appl. No.: 10/537,201

(22) PCT Filed: Dec. 2, 2003
(86) PCT No.: PCT/EP03/50928

§ 371 Date: Dec. 20, 2005

(30) Foreign Application Priority Data
Dec. 3, 2002 (EP) 02080054.6

(51) Int. Cl. C12N 15/74; C12N 1/21; C07H 21/04

(52) U.S. Cl. 435/471; 536/23.2; 435/252.2

(57) ABSTRACT

The present invention provides an isolated polynucleotide comprising the kstD promoter from Rhodococcus erythrypolis. The polynucleotide can very advantageously be used as a controllable transcription activator. Said controlling function can be provided by providing said isolated polynucleotide with a nucleotide sequence encoding a transcription regulator of said promoter. In the present invention, such a transcription regulator may be externally induced, such as by introduction of steroidal compounds. In an alternative embodiment of the present invention the isolated polynucleotide may comprise the kstR gene or a homologue or a functional part thereof as the transcription regulator of the kstD promoter.

### FIELD OF THE INVENTION

The invention concerns a promoter from Rhodococcus, more specifically Rhodococcus erythropolis, its regulation and the use of this promoter and its regulation as an expression system in heterologous applications.

### BACKGROUND OF THE INVENTION

Actinomycetes, and especially bacteria of the genus Rhodococcus are renowned for their ability to metabolise complex molecules. Several species of Rhodococcus are able to degrade fuel, benzene, and even TNT and they are therefore widely studied in the field of microbiology which concerns the biochemical pathways and cell factories. Among the micro-organisms which oxidize natural and anthropogenic hydrocarbons and which are active participants in biogeochemical processes of the biosphere, e.g. contributing to producing a hydrocarbon-free atmosphere for the Earth, the genus Rhodococcus takes a predominant place.

Several Rhodococcus species also degrade natural phytosterols, which proceeds via the formation of steroids as pathway intermediates. These steroids may in turn be used as precursors in the production of pharmaceutically active compounds.

In order to produce pharmaceutical precursor compounds as pathway intermediates of microbes in high amounts, production strains are routinely transformed to optimise the expression of the genes of interest and/or block certain metabolic routes in order to achieve accumulation of the intermediates. Such transformation often involves the heterologous expression of proteins. With the increased use of Rhodococcus and other Actinomycetic bacteria (such as Mycobacterium, Arthrobacter, Nocardia, Corynebacterium and Brevibacterium species) for expression of heterologous proteins, there is a growing need for improved regulation of such expression and for molecular tools.

Presently, mutant strains with desired properties are isolated by classical mutagenesis, such as UV irradiation, but these strains are often inadequate in industrial processes due to genetic instability and/or low bioconversion efficiencies. Molecularly defined (constructed) mutants would present important advantages over mutants generated by classical mutagenesis. Constructed mutants are genetically more stable and the introduced mutations represent well-defined genetic modifications. Construction of genetically engineered strains by transformation may also make the use of chemical agents to block certain pathways obsolete. Chemical agents used to block enzyme activity mostly are often not reaction specific and may inhibit other important enzymatic reactions, which may have negative effects on bioconversion efficiency. The use of defined mutants by genetic engineering would overcome such problems.

An important enzyme in steroid metabolism, which can, for instance, be found in Rhodococcus erythropolis, is 3-ketosteroid Δ¹-dehydrogenase (KSTD1, EC 1.3.99.4) the gene of which resides in the so-called kstD1 locus (van der Geize, R. et al. 2000. Appl. Environm. Microbiol. 66: 2029-2036). Although it is known that molecular organization of steroid catabolic genes may differ between different Rhodococcus species, homologues of this gene have been found in several other bacteria, such as Arthrobacter simplex, Pseudomonas spp., Nocardia restrictus, Nocardia corallina, Nocardia opaca and Mycobacterium fortuitum. The sequence of the kstD gene of Rhodococcus erythropolis strain SQ1 has been disclosed by Van der Geize et al. in WO 01/31050 and is depicted in SEQ ID NO:1.

From the same bacterial strain an isoenzyme KSTD2 with its corresponding gene kstD2, is known. Disruption of the kstD1 gene has shown not to abolish 3-ketosteroid Δ¹-dehydrogenase (KSTD) activity completely and activity remains due to the presence of the isoenzyme (Van der Geize et al., 2002. Microbiology 148:3285-3292; WO 01/31050).

KSTD activity is essential for steroid nucleus degradation and kstD gene inactivation is needed to accumulate steroid intermediates. Inactivation of genes is a powerful tool for analyzing gene function and for introducing metabolic blocks. Gene disruption with a non-replicative vector carrying a selectable marker is the commonly used method for gene inactivation.

It was found that wild-type RSTD activity in gene disruption mutant R. erythropolis SDH420 can be induced by the application of 3-keto-Δ⁴-steroids, such as 4-androstene-3,17-dione, indicating the presence of a steroid-dependent regulatory mechanism. Upstream of the hstd gene locus, a gene (ORF2) was identified whose function was hitherto unknown, but was described as a putative regulatory gene carrying the consensus sequence of repressor proteins of the TetR family (Van der Geize, R. et al. 2000. Appl. Environ. Microbiol. 66:2029-2036).

It has now been found that a promoter for the kstD1 gene resides in the kstD locus of Rhodococcus erythropolis and that this promoter is regulated through repression with the gene product of ORF2 of the bacterium, denominated kstR hereafter. It has now also been found that this repression of the kstD promoter by the kstR gene-product can be overcome by the induction of expression with steroidal compounds. This property of the combination of the kstD gene-kstR repressor system makes it particularly fit for expression of heterologous proteins in bacteria such as those of the family of Actinomycetes.

### SUMMARY OF THE INVENTION

In one aspect, the invention relates to an isolated polynucleotide comprising a promoter from Rhodococcus erythropolis, characterised in that said promoter is the kstD promoter.

The polynucleotide can very advantageously be used as a controllable transcription activator. Said controlling function can be provided by providing said isolated polynucleotide with a nucleotide sequence encoding a transcription regulator of said promoter. In the present invention, such a transcription regulator may be externally induced, such as by introduction of steroidal compounds.

In an alternative embodiment of the present invention the isolated polynucleotide may comprise the kstR gene or a homologue or a functional part thereof as the transcription regulator of the kstD promoter.

Since the isolated polynucleotide of the invention can very advantageously be used as a heterologous expression system, the polynucleotide of the invention may further comprise a nucleotide sequence encoding a polypeptide that is operably linked to said promoter.

In order to provide for selectable traits in the bacteria into which the expression system is transferred, the polynucleotide may further comprise such sequences that encode selectable markers, counter-selectable markers and/or reporter genes.

In another aspect, the invention relates to a recombinant vector comprising an isolated polynucleotide of the invention. Such a recombinant vector suitably comprises nucleotide sequences that represent multiple cloning sites.

The present invention also relates to a method for constructing a genetically modified strain of a micro-organism which micro-organism lacks the ability to degrade the steroid nucleus, the method comprising producing a polynucleotide according to the present invention and transforming the said strain with said polynucleotide.

In another aspect the invention relates to a host cell transformed with the recombinant vector of the invention. Said host cell is preferably a bacterium from the order of Actinomycetales. Very suitable host cells are bacteria belonging to the families of Actinomycetaceae, Corynebacterineae, Mycobacteriaceae, Nocardiaceae, Brevibacteriaceae, or Micrococcaceae and in particular those of the genus Rhodococcus.

In another aspect, the invention relates to a method for producing a desired protein in a host cell, comprising transforming a host cell with a recombinant vector of the invention.

In another aspect, the invention relates to a microbial expression system comprising a polynucleotide of the invention.

In yet another aspect, the invention relates to a method for constitutive expression of a protein of interest comprising transforming a host cell with a polynucleotide construct wherein the expression of the coding region of said protein is under control of the kstD promoter.

In another aspect, the invention relates to a use of a steroid for the induction of expression of a heterologous protein, which expression is under control of the kstD promoter, said steroid lifting the repressor function exerted by the kstR gene product.

### DESCRIPTION OF THE FIGURES

FIG. 1 is a schematic representation of the construction of mutagenic plasmid pREG104 for kstR unmarked gene deletion.

FIG. 2 is a schematic representation of the Rhodococcus expression vector pRESX derived from pRESQ (Van der Geize, R. et al. 2002. Mol. Microbiol. 45:1007-1018). Closed solid curved bar indicates the kstD promoter region. Open solid curved bar indicates Rhodococcus genes encoding autonomous replication. aphII encodes kanamycine resistance.

### DETAILED DESCRIPTION OF THE INVENTION

The term “polynucleotide” as used herein refers to a polymeric form of nucleotides of any length, either ribonucleotides or deoxyribonucleotides. Thus, this term includes double- and single-stranded DNA and RNA.

The term “recombinant polynucleotide” as used herein intends a polynucleotide of genomic, cDNA, semisynthetic, or synthetic origin which, by virtue of its origin or manipulation: (1) is not associated with all or a portion of a polynucleotide with which it is associated in nature; or (2) is linked to a polynucleotide other than that to which it is linked in nature; or (3) does not occur in nature.

“Transformation” and “transforming”, as used herein, refers to the insertion of an exogenous polynucleotide into a host cell, irrespective of the method used for the insertion, for example, direct uptake, transduction, f-mating or electroporation. The exogenous polynucleotide may be maintained as a non-integrated vector, for example, a plasmid, or alternatively, may be integrated into the host cell genome.

By “host cell” is meant a cell which contains a vector and supports the replication and/or expression of the vector. Host cells may be prokaryotic cells such as E. coli, or eukaryotic cells such as yeast, insect, amphibian, or mammalian cells. Preferably, host cells are bacterial cells of the order of Actinomycetales.

As used herein, the term “operably linked” refers to a juxtaposition wherein the components so described are in a relationship permitting them to function in their intended manner. A control sequence “operably linked” to another control sequence and/or to a coding sequence is ligated in such a way that transcription and/or expression of the coding sequence is achieved under conditions compatible with the control sequence. Generally, operably linked means that the nucleic acid sequences being linked are contiguous and, where necessary to join two protein coding regions, contiguous and in the same reading frame.

As used herein “promoter” is a DNA sequence that directs the transcription of a (structural) gene. Typically, a promoter is located in the 5′region of a gene, proximal to the transcriptional start site of a (structural) gene. If a promoter is an inducible promoter, then the rate of transcription increases in response to an inducing agent. In contrast, the rate of transcription is not regulated by an inducing agent if the promoter is a constitutive promoter.

The term “polypeptide” refers to a polymer of amino acids and does not refer to a specific length of the product; thus, peptides, oligopeptides, and proteins are included within the definition of polypeptide. This term also does not refer to or exclude post-expression modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as the modifications known in the art, both naturally occurring and non-naturally occurring.

As used herein, “heterologous” in reference to a nucleic acid is a nucleic acid that originates from a foreign species, or, if from the same species, is by deliberate human intervention at a different native genomic locus than in the native state. For example, a promoter operably linked to a heterologous structural gene is from a species different from that from which the structural gene was derived, or, if from the same species, the promoter and the gene are not operably linked in nature. A heterologous protein may originate from a foreign species or, if from the same species, is produced through expression from a heterologous nucleic acid.

A “repressor protein” or “repressor” is a protein that is able to recognize and bind to a nucleotide sequence that is contained in a DNA sequence (operator) that is located 5′-ward of a structural gene. The binding of a repressor protein with its cognate operator results in the inhibition of the transcription of the structural gene.

An “enhancer” is a DNA regulatory element that can increase the efficiency of transcription, regardless of the distance or orientation of the enhancer relative to the start site of transcription.

The term “isolated” refers to material, such as a nucleic acid, which is substantially or essentially free from components that normally accompany or interact with it as found in its naturally occurring environment. An isolated DNA molecule is a fragment of DNA that has been separated and that is no longer integrated in the genomic DNA of the organism from which it is derived.

The term “expression” refers to the biosynthesis of a gene product.

An “expression vector” is a DNA molecule comprising a gene that is expressed in a host cell. Typically, gene expression is placed under the control of certain regulatory elements, including constitutive or inducible promoters, regulatory elements and/or enhancers. Such a gene is said to be “operably linked to” the regulatory elements and its expression is said to be “under control of” the regulatory elements.

The term “selectable marker” refers to a polynucleotide sequence encoding a metabolic trait which allows for the separation of transgenic and non-transgenic organisms and mostly refers to the provision of antibiotic resistance. A selectable marker is for example the aphII encoded kanamycin resistance marker.

The term “counter-selectable marker” refers to a polynucleotide sequence whose expression is lethal, instead of giving rise to resistance as is often the case for selectable markers. A counter-selectable marker is for example the sacb gene encoding B. subtilis levansucrase the expression of which is lethal in the presence of sucrose.

As used herein, the term “reporter gene” means a gene that encodes a gene product that can be identified. Reporter genes include, but are not limited to, chloramphenicol acetyl transferase, [beta]-galactosidase, luciferase and green fluorescence protein. Identification methods for the products of reporter genes include, but are not limited to, enzymatic assays and fluorimetric assays. Reporter genes and assays to detect their products are well known in the art and are described, for example in Current Protocols in Molecular Biology, eds. Ausubel et al., Greene Publishing and Wiley-Interscience: New York (1987) and periodic updates.

The sequence denoted as ORF2 of Rhodococcus, which is depicted in SEQ ID NO:4, has been deemed to be part of the chromosomal gene cluster also harbouring the kstD1 gene (Van der Geize, R. et al. 2000. Appl. Environ. Microbiol. 66:2029-2036), where it is said to encode a TetR type of repressor protein (denominated kstR). However, the circumstances under which the repressor function is exercised or lifted have never been disclosed up till now. Also, the relation between the repressor function of kstR and the promoter of the kstD1 gene has hitherto not been established.

In Arthrobacter simplex a similar genomic composition of the kstD gene and a putative repressor coding ORF (denominated kdsR) has been described (Molnar, I. et al. 1995. Mol. Microbiol. 15:895-905). In this case, no relation between repressor protein and expression of the steroid enzyme has been established.

The promoter region of the kstD1 gene has not been exactly determined. The region between the start codons of the kstD1 gene and the start of the kstR gene (which lies in the reverse order compared to the kstD1 gene) is a sequence of about 158 basepairs, which contains the promoter for the kstD1 gene and presumably also the promoter for kstR. In case this promoter would be working bidirectionally, the expression of gene encoding the repressor protein can also be driven by the kstD1 promoter.

The promoter according to the invention is the promoter driving expression of the kstD gene in Rhodococcus and it preferably comprises the nucleotide sequence of 158 base pairs according to SEQ ID NO: 3 or a shortened version thereof (e.g. deleted at the 5′ end) which still possesses the functional capacity of a promoter, i.e. to drive the expression of a protein which expression it controls. How to arrive at promoter deletion mutants is well known in the art and also the experimentation needed to identify promoter activity for such a deletion mutant comprises no undue burden and is well known to a person skilled in the art. Techniques for polynucleotide manipulation useful for the practice of the present invention are described in a variety of references, such as Molecular Cloning: A Laboratory Manual, 2nd d., Vol. 1-3, eds. Sambrook et al. Cold Spring Harbor Laboratory Press (1989) or Current Protocols in Molecular Biology, eds. Ausubel et al., Greene Publishing and Wiley-Interscience: New York (1987) and periodic updates thereof.

One skilled in the art would know methods for identifying active fragments of kstD promoter, which methods could include, for example, the measurement of transcription of mRNA or the expression of a polypeptide from a reporter gene which requires the addition of a functional promoter. To determine the presence of active kstD promoter fragments that are capable of controlling transcription and/or expression of the coding sequence to which it is operably linked, the person skilled in the art will readily understand that a promoter functionality test can be performed therewith. Such a test may for instance comprise the operable linking of a promoter of the invention and a reporter gene in a vector, bringing the vector in a suitable host, exposing the host to conditions suitable for expression and determining the presence of the reporter gene product in order to determine promoter functionality.

While the nucleotide sequence of the promoter (including promoter elements) is given in SEQ ID NO:3, it is recognized that nucleotide substitutions can be made which do not affect the promoter or promoter element function.

One skilled in the art would recognize that point mutations and deletions can be made to the kstD promoter sequences disclosed herein without altering the ability of the sequence to activate transcription. In addition, active fragments of the kstD promoter can be obtained. Similar methods can be used for identifying other active fragments of the kstD promoter. Other methods for identifying an active fragment of the kstD promoter are routine and well known in the art. For example, overlapping fragments of the kstD promoter can be synthesized and cloned into a suitable expression vector to determine active kstD promoter fragments. Similarly, point mutations can be introduced into the disclosed kstD promoter sequences using, for example, site-directed mutagenesis or by synthesizing sequences having random nucleotides at one or more predetermined positions.

The invention includes as an embodiment an isolated polynucleotide comprising a kstD promoter or active fragment thereof. These isolated polynucleotides contain less than about 50%, preferably less than about 70%, and more preferably less than about 90% of the chromosomal genetic material with which the kstD promoter is usually associated in nature. An isolated polynucleotide “consisting essentially of” a kstD promoter lacks other promoters derived from the chromosome on which kstD is located. This terminology of “isolated” and “consisting essentially of” is analogously applicable to the kstR repressor element. For example, an isolated polynucleotide consisting essentially of a kstR repressor lacks polynucleotide material such as enhancers or promoters, respectively, located on the chromosome on which kstR is located.

Isolated polynucleotides comprised of or consisting essentially of a kstD promoter, and coding for a kstR repressor or active fragments thereof, may be prepared by techniques known in the art (e.g., Sambrook, et al.). These techniques include, for example, using the sequence information provided herein to provide primers and probes to amplify by PCR specific regions of kstD genomic clones, or by chemical synthesis, or by recombinant means.

A recombinant polynucleotide comprised of a kstD promoter or active fragment thereof, as well as those which may be comprised of other kstD transcription regulatory elements described herein, may be prepared by any technique to those of skill in the art using the sequence information provided herein.

In the experimental section the promoter is shown to be regulated by the repressor protein, which is presumed to bind to the promoter and thus to inhibit the expression of the protein which it controls. A recombinant polynucleotide comprised of a kstD promoter may also be comprised of a coding sequence for the kstR repressor (such as depicted in SEQ ID NO:4) causing repression of kstD-promoted gene transcription and providing a regulation mechanism that can be lifted by exposing the cells to an inducer such as described below.

A recombinant polynucleotide comprising a kstD promoter may also comprise a coding sequence to which the promoter is operably linked, causing transcription of the coding sequence under the control of the promoter. Coding sequences may encode either homologous or heterologous polypeptides. However, they may also encode other moieties which are desirable in their transcribed form. For example, coding sequences may encode, inter alia, decoy polynucleotides that bind to transcription factors, anti-sense RNAs, and a variety of polypeptides that are of interest (e.g. viral proteins to serve as intracellular vaccines, proteins that serve as markers, etc.), polypeptides for commercial purposes that are to be expressed in cells that express kstD proteins, and particularly proteins that are of use in regulation of cell metabolism and production of pharmaceutical precursors.

For extracellular expression of proteins under control of the promoter a signal sequence can be inserted between the promoter and the DNA coding for the gene of interest. Such a signal sequence is provided to allow targeting of proteins to specific cellular compartments. Preferably this signal sequence is the signal sequence of the gene coding for cholesterol oxidase as present in R. equi and as deposited at Genbank under accession number AJ242746 (see also Navas, J. et al. 2001. J. Bacteriol. 183:4796-4805).

The promoter can be used in any host cell, but preferably in a prokaryote host cell, more preferably a bacterium from the order of Actinomycetales, such as those bacteria belonging to families such as Actinomycetaceae, Corynebacterineae, Mycobacteriaceae, Nocardiaceae, Brevibacteriaceae, Micrococcaceae and the like. More preferably it will be a bacterium of the genus Rhodococcus, Mycobacterium, Arthrobacter, Nocardia, Corynebacterium or Brevibacterium. Most preferably it is a bacterium of the genus Rhodococcus such as a bacterial strain of the species Rhodococcus aetherovorans, Rhodococcus coprophilus, Rhodococcus equi, Rhodococcus erythreus, Rhodococcus erythropolis, Rhodococcus fascians, Rhodococcus globerulus, Rhodococcus jostii, Rhodococcus koreensis, Rhodococcus maanshanensis, Rhodococcus marinonascens, Rhodococcus opacus, Rhodococcus percolatus, Rhodococcus pyridinivorans, Rhodococcus rhodnii, Rhodococcus rhodochrous, Rhodococcus rubber, Rhodococcus tukisamuensis, Rhodococcus wratislaviensis, Rhodococcus zopfii and the like.

Also part of the invention is a bacterial host cell which is equipped with the promoter, without having the gene for the repressor protein. Preferably this host cell is the bacterium Rhodococcus erythropolis RG10 as deposited under number DSM 15231 with the DSMZ-Deutsche Sammlung von Mikroorganismen und Zellkulturen at Oct. 9, 2002. In this bacterium, as is shown in Example 1, the gene coding for the suppressor protein has been deleted.

Such a host cell, in which the suppressor gene has been deleted can be used for the expression of proteins. Preferably (although it may be that the host cell still has its endogenous kstD promoter) a vector harbouring the kstD promoter of the invention which controls the expression of a protein of interest should the be introduced in the cell. Constructing such a vector and transformation or transfection of such a vector into the host cell is common practice for those skilled in the art. In this way, as is shown in Example 1, the kstD promoter will be unrepressed and act as a constitutive promoter.

A host cell according to the invention comprising the isolated kstD promoter or an active fragment thereof is understood to include the progeny of the original cell which has been transformed. It is understood that the progeny of a single parental cell may not necessarily be completely identical in morphology or in genomic or total DNA complement as the original parent, due to natural accidental, or deliberate mutation.

It is recognized that specific nucleotides or regions within the kstD promoter elements other than kstR may be identified as necessary for regulation. These regions of nucleotides may be located by fine structural dissection of the elements by analyzing the functional capacity of a large number of promoter mutants. Single base pair mutations can be generated utilizing polymerase chain reaction (PCR) technology. U.S. Pat. No. 4,683,202. Mutated promoter regions can be cloned back into reporter constructs using standard techniques and evaluated by transfection into appropriate cells and assayed for reporter gene function. This analysis will also identify nucleotide changes which do not affect promoter function.

A further aspect of the invention is to use the repressor and its inducibility for controlled expression by providing a cell with a sequence that codes for the repressor protein and a sequence coding for a protein that needs to be controllably expressed, where that sequence is operably linked to the kstD promoter. The sequence for the kstR repressor protein may be encompassed on the same expression construct as the kstD promoter-gene of interest construct, but it may also be on a different construct. It is also envisaged that the host cell already contains the repressor gene, either located on a plasmid or on the chromosome. Then, the expression system is established by transforming such a host cell with a construct harbouring the kstD promoter. Similarly, the host cell may already contain the promoter controlling the expression of a gene of interest. Addition of the gene coding for the repressor protein then would stop expression of the gene of interest when the repressor protein is produced and expression can be induced again by lifting the repressor function. Also, the expression of the kstR repressor sequence may be under the control of a constitutive promoter.

A further method for controlling expression of a gene of interest by the kstD-kstR system is by replacing the kstD gene which is normally under control of the kstD promoter by inserting the coding sequence of a gene of interest in situ behind the kstD promoter. This can be accomplished by techniques which are commonly known within the art, such as homologous recombination and/or use of recombinases and their recognition sites such as the cre-lox system.

The repression of the kstD promoter exerted by the kstR gene product can be lifted by addition of a steroid compound, with 3-keto-Δ⁴-functionality. In particular such compounds as 4-androstene-3,17-dione (AD), 1,4-androstadiene-3,17-dione (ADD), estr-4-ene-3,17-dione, testosterone, progesterone, nordione, 7α-methyl-nordione, 11-methylene-nordione, but also such compounds as pregnenolone and stanolone (17β-OH-5α-androstane-3-on), 19-OH-7-dehydro-androstene-3,17-dione and 9α-hydroxy-4-androstene-3,17-dione (9OHAD) are able to lift kstD promotor repression.

Alternative regulatory compounds may also be identified. For instance, cells expressing products of reporter genes under the control of a kstD promoter are useful for identifying agents that regulate the activity of a kstD promoter. Thus, host cells expressing a reporter gene product under the control of a kstD promoter are useful for screening and it is a further object of the invention to provide a method for identifying compounds that regulate the activity of a kstD promoter. The method includes exposing a cell containing a kstD promoter to at least one compound whose ability to modulate the activity of a kstD promoter is sought to be determined. The cells are then monitored for changes caused by the modulation.

### EXAMPLES

### Example 1

### Constitutive Expression of kstD Following kstR Unmarked Gene Deletion

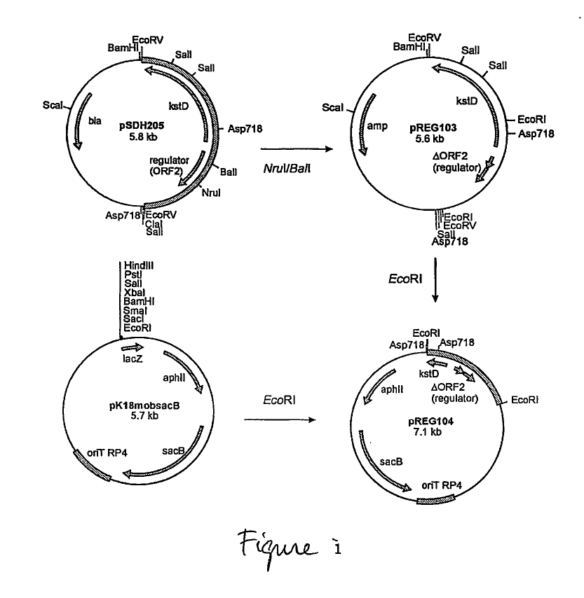

Mutagenic plasmid pREG104 was constructed for unmarked gene deletion of kstR, the gene encoding a transcription regulator of the kstD gene (encoding 3-ketosteroid Δ¹-dehydrogenase KSTD1) in Rhodococcus erythropolis SQ1 (FIG. 1). Briefly, pSDH205 (Van der Geize, R. et al. 2000. Appl. Environ. Microbiol. 66:2029-2036) was digested with restriction enzymes NruI and BalI followed by self-ligation, resulting in plasmid pREG103. An EcoRI DNA fragment of pREG103, containing the kstR gene deletion was subsequently cloned into EcoRI digested pK18mobsacB vector, resulting in pREG104. Unmarked kstR gene deletion mutant R. erythropolis RG10 was isolated from R. erythropolis SQ1 using pREG104 via the sacB counter-selection method as described (Van der Geize R. et al. 2001. FEMS Microbiol. Lett. 205:197-202). Genuine kstR gene deletion was confirmed by the polymerase chain reaction (PCR) using forward primer (REG-REV) 5αGGCGACGTTGCCGAGAATT 3′ and reverse primer (REG-REV) 5′TCAGTGTCGTGAGAGATTCA 3′. A PCR amplicon of 618 bp was obtained with parent strain SQ1 genomic DNA (control). With genomic DNA of kstR gene deletion mutant strain RG10 the amplicon was reduced to 393 bp, confirming kstR gene deletion.

Constitutive KstD1 expression was checked by growing cells of mutant strain RG10 and parent strain SQ1 in glucose (20 mM) mineral medium (1 g·l⁻¹ NH₄NO₃, 0.25 g·l⁻¹ K₂HPO₄, 0.25 g·l⁻¹ MgSO₄.7H₂O, 5 mg·l⁻¹ NaCl, 5 mg·l⁻¹ FeSO₄.7H₂O (pH 7.2)) for 3 days at 30° C. followed by steroid induction for 5 hours (0.5 g·l⁻¹ 4-androstene-3,17-dione (AD)). As a control, cell cultures without steroid induction were used. AD was solubilized in DMSO (50 mg·ml⁻¹) and added to the autoclaved medium. Cell pellets (30 min; 7,300×g; 4° C.) were washed with 200 ml phosphate buffer (KH₂PO₄ 2.72 g·l⁻¹; K₂HPO₄ 3.48 g·l⁻¹; MgSO₄.7H₂O 2.46 g·l; pH 7.2). Washed cell suspensions (5 ml) were disrupted by double passage through a French pressure cell (140 Mpa). Cell extracts were centrifuged for 20 min at 25,000×g to remove cell debris. Expression of kstD was checked by native polyacrylamide gel electrophoresis (PAGE) stained for KSTD activity (Van der Geize, R. et al. 2000. Appl. Environ. Microbiol. 66:2029-2036) (Table 1). A KSTD1 activity band was found with cell free extracts prepared from non-induced cells of strain RG10, indicating that kstR gene deletion results in constitutive expression of the kstD gene (Table 1).

TABLE 1
Constitutive kstD expression upon kstR unmarked gene deletion
checked by native PAGE stained for KSTD1 activity.

| | |
|---|---|
| steroid | |
| induction | kstD expression |

| (AD or | | kstR mutant strain |
|---|---|---|
| 9OHAD) | parent strain SQ1 | RG10 |
| − | − | + |
| + | + | + |

### Example 2

### Constitutive Expression of KstD2 for Microbial Steroid Δ¹-dehydrogenation

A kstR gene deletion mutant strain of R. erythropolis RG9 (Van der Geize, R. et al. 2002. Mol. Microbiol. 45:1007-1018) was constructed, designated R. erythropolis RG17, using pREG104 (FIG. 1, see example 1). Strain RG17 thus is a kstD kstD2 kshA1 kstR quadruple gene deletion mutant, lacking 3-ketosteroid Δ¹-dehydrogenase (KSTD1 and KSTD2) and 3-ketosteroid 9α-hydroxylase (KSE) activities, in addition to the transcription regulator of the kstD promoter. Due to the kstD kstD2 kshA phenotype of this mutant, strain RG17 is completely blocked in metabolizing 4-androstene-3,17-dione (AD), 1,4-androstadiene-3,17-dione (ADD) and 9α-hydroxy-4-androstene-3,17-dione (9OHAD).

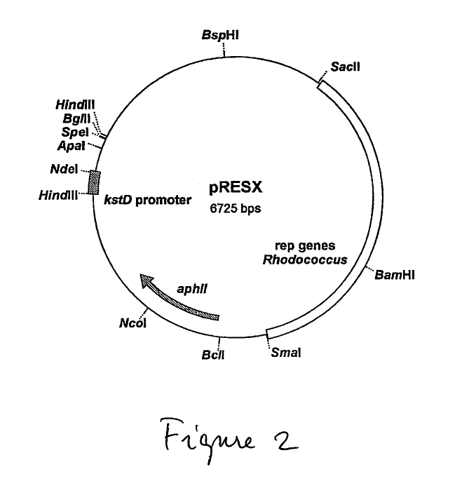

A Rhodococcus expression vector was constructed for the expression of genes under control of the kstD promoter of R. erythropolis SQ1 (Van der Geize, R. et al. 2000. Appl. Environ. Microbiol. 66:2029-2036). Using the kstD promoter, expression of genes in R. erythropolis mutant strains harboring a kstR gene deletion will be constitutive due to the absence of the repressor of kstD expression. The kstD promoter region (158 bp) was isolated from R. erythropolis SQ1 chromosomal DNA by PCR amplification (25 cycles: 30 s 95° C., 30 s 64° C., 30 s 72° C., using Taq polymerase) using forward primer 5′ATAAAGCTTATCGATTATGTGTCCCGGCCGCGAAC3′ and reverse primer 5′ATAGGTACCATATGTGCGTCCTTACTCCAAGAGGG3′. A NdeI site (underlined) was incorporated in the amplicon to be able to clone genes of interest precisely at the ATG startcodon of the kstD gene. The amplicon (175 bp) was blunt-ligated into the unique SnaBI restriction site of shuttle vector pRESQ (Van der Geize, R. et al. 2002. Mol. Microbiol. 45:1007-1018) and the resulting Rhodococcus expression vector was designated pRESX (FIG. 2).

The kstD2 gene, encoding the KSTD2 isoenzyme in R. erythropolis SQ1, was isolated from chromosomal DNA of parent strain SQ1 by PCR (conditions: see above), using forward primer 5′ GCGCATATGGCTAAGAATCAGGCACCC 3′ (NdeI site underlined) and reverse primer 5′ GCGGGATCCCTACTTCTCTGCTGCGTGATG 3′ (BamHI site underlined). The introduced NdeI and BamHI sites were used to ligate the kstD2 amplicon into NdeI/BglII digested pRESX vector. The resulting plasmid was designated pRESX-KSTD2.

Plasmid pRESX-KSTD2 was introduced into R. erythropolis strain RG17 by electrotransformation (Van der Geize, R. et al. 2000. Appl. Environ. 15 Microbiol. 66:2029-2036) and one transformant was used for AD biotransformation. Biotransformation of AD into ADD by NSTD2 was performed with cultures grown in 100 ml YG15 (15 g·l⁻¹ yeast extract, 15 g·l⁻¹ glucose) medium at 28° C. (200 rpm) in the presence of kanamycine (200 μg·ml⁻¹). After growth till late exponential phase (OD₆₀₀ of 5 to 9), AD (1 g·l⁻¹ in 0.1% [vol/vol] Tween80) was added and AD biotransformation into ADD was followed for several days. For HPLC analysis, culture samples were diluted 5 times with methanol/water (70:30) and filtered (0.45 m). Steroids were analyzed by HPLC (with a 250- by 3-mm reversed phase Lichrosorb 10RP18 column [Varian Chrompack International, Middelburg, The Netherlands], UV detection at 254 nm, and a liquid phase of methanol-water [60:40] at 35° C.).

Biotransformation experiments with cells of R. erythropolis strain RG17, harboring pRESX-KSTD2, showed the presence of KSTD2 steroid Δ¹-dehydrogenase activity resulting in biotransformation of AD into ADD to near completion. In contrast, Rhodococcus mutants with wild type KSTD1 and KSTD2 isoenzymes, but blocked in the KSH reaction, convert AD into ADD in yields usually not exceeding 50%, probably due to regulatory mechanisms (Van der Geize, R. et al. 2002. Mol. Microbiol. 45:1007-1018).

### Example 8

### Expression of kshA Isogene kshA2 Complements the kshA Mutant Phenotype

A homologue of the kshA gene of R. erythropolis SQ1 (Van der Geize, R. et al. 2002. Mol. Microbiol. 45:1007-1018) was identified following nucleotide sequencing of DNA fragments isolated by complementation experiments of UV-induced Rhodococcus mutants. R. erythropolis SQ1 contains at least two kshA isogenes, which were designated kshA and kshA2.

R. erythropolis RG2, a kshA gene deletion mutant of R. erythropolis SQ1 (Van der Geize, R. et al. 2002. Mol. Microbiol. 45:1007-1018), does not show growth on mineral agar plates (1 g·l⁻¹ NH₄NO₃, 0.25 g·l⁻¹ K₂HPO₄, 0.25 g·l⁻¹ MgSO₄.7H₂O, 5 mg·l⁻¹ NaCl, 5 mg·l⁻¹ FeSO₄.7H₂O (pH 7.2), 1.5% agar) supplemented with AD (0.25 g·l⁻¹) as sole carbon and energy source. Thus, kshA2 is not expressed under these growing conditions in R. erythropolis RG2.

The kshA2 gene was placed under control of the kstD promoter in pRESX. In order to achieve this, the kshA2 gene was amplified from R. erythropolis chromosomal DNA as template by PCR using forward primer 5′GGCCATATGTTGACCACAGACGTGACGACC 3′ (NdeI site underlined) and reverse primer 5′ GCCACTAGTTCACTGCGCTGCTCCTGCACG 3′ (SpeI site underlined). The obtained kshA2 amplicon was first ligated into EcoRV digested pBlueScript (II)KS (pKSH311) and subsequently subcloned as a NdeI/SpeI fragment into NdeI/SpeI digested pRESX, resulting in pKSH312.

Plasmid pKSH312 was introduced into R. erythropolis RG2 by electrotransformation and the resulting transformants were replica plated onto mineral agar medium containing 0.25 g·l⁻¹ of AD as sole carbon and energy source. All transformants were able to grow on AD mineral medium, indicating functional expression of kshA2 under control of the kstD promoter and complementation of the kshA mutant phenotype.

### Example 4

### Inducing Steroids and Constitutive Expression

In order to assess which steroids are able to induce the repressor-promoter system of the KstD gene, cell cultures of both R. erythropolis SQ1 (wildtype) and R. erythropolis RG10 (kstR-mutant) as described above were tested under inducing conditions with 1,4-androstadiene-3,17-dione (ADD), testosterone, progesterone, nordione, estron, 7α-methyl-nordione, 11-methylene-nordione, stanolone (17βOH-5α-androstane-3-one), 19OH-7-dehydro-androstene-3,17-dione and pregnenolone were tested. 4-androstene-3,17-dione (AD) induction served as a positive control.

From induced cultures, cell free extracts were prepared as indicated above and tested for KSTD activity using DCPIP as an electron acceptor and AD as a substrate. With the exception of estron, it was found that all steroids tested were able to induce KSTD activity in R. erythropolis SQ1. The level of activity was not the same for all steroids tested. Controls on native gels confirmed that KSTD1 activity was indeed induced in all positive cases.

Further, it was investigated whether KSTD1 was constitutively expressed in R. erythropolis RG10. Cell free extracts were prepared from an AD-induced cell culture and from a non-induced cell culture. These extracts were tested for KSTD activity using DCPIP as an electron acceptor and AD as a substrate. For reasons of comparison, the same procedure was performed with R. erythropolis SQ1.

It was found that in both the induced as well as the non-induced culture of R. erythropolis RG10, KSTD activity was present. In R. erythropolis SQ1, on the other hand, KSTD activity was only detected in the AD-induced culture. Controls on native gels confirmed that KSTD1 activity was indeed induced in all positive cases.

| | | |
|---|---|---|
| 0-1 | Form - PCT/RO/134 (EASY) | |
| | Indications Relating to Deposited | |
| | Microorganism(s) or Other Biological | |
| | Material (PCT Rule 13bis) | |
| 0-1-1 | Prepared using | epoline ® online filing PCT plug-in |
| | | (updated 02.12.2003) |
| 0-2 | International Application No. | |
| 0-3 | Applicant's or agent's file reference | 2002.744_WO |
| 1 | The indications made below relate to | |
| | the deposited microorganism(s) or | |
| | other biological material referred to in | |
| | the description on: | |
| 1-1 | page | 13 |
| 1-2 | line | 12 |
| 1-3 | Identification of Deposit | |
| 1-3-1 | Name of depositary Institution | DSMZ-Deutsche Sammlung von |
| | | Mikroorganismen und Zellkulturen GmbH |
| 1-3-2 | Address of depositary Institution | Mascheroder Weg 1b, D-38124 |
| | | Braunschweig, Germany |
| 1-3-3 | Date of deposit | 09 Oct. 2002 (09.10.2002) |
| 1-3-4 | Accession Number | DSMZ 15231 |
| 1-4 | Additional Indications | NONE |
| 1-5 | Designated States for Which | all designated States |
| | Indications are Made | |
| 1-6 | Separate Furnishing of Indications | NONE |
| | These Indications will be submitted to the | |
| | International Bureau later | |

| | |
|---|---|
| 0-4 | This form was received with the |
| | International application: |
| | (yes or no) |
| 0-4-1 | Authorized officer |

| | |
|---|---|
| 0-5 | This form was received by the |
| | International Bureau on: |
| 0-5-1 | Authorized officer |

1. A recombinant polynucleotide comprising the kstD promoter from Rhodococcus.

2. The recombinant polynucleotide according to claim 1, wherein said Rhodococcus is Rhodococcus erythropolis.

3. The recombinant polynucleotide according to claim 1, wherein the promoter comprises nucleotides 1-158 from the sequence of SEQ ID NO:3 or a functional part thereof.

4. The recombinant polynucleotide according to claim 2, further comprising a nucleotide sequence encoding a transcription regulator of said promoter.

5. The recombinant polynucleotide according to claim 4, wherein the expression of said nucleotide sequence is controlled by steroidal compounds.

6. The recombinant polynucleotide according to claim 5, wherein said regulator comprises the kstR gene or a homologue or a functional part thereof.

7. The recombinant polynucleotide according to claim 6, further comprising a nucleotide sequence encoding a heterologous polypeptide that is operably linked to said promoter.

8. The recombinant polynucleotide according to claim 7, further comprising at least one nucleotide sequence selected from the group consisting of a selectable marker, a counter-selectable marker and a reporter gene.

9. The recombinant polynucleotide according to claim 7, further comprising a signal sequence.

10. A recombinant vector comprising the recombinant polynucleotide according to claim 7.

11. A recombinant vector according to claim 10, further comprising a nucleotide sequence having multiple cloning sites.

12. A host cell transformed with the recombinant vector according to claim 10.

13. The host cell according to claim 12, wherein said host cell is a bacterium from the order of Actinomycetales.

14. The host cell according to claim 13, wherein said host cell is selected from bacteria belonging to the families of Actinomycetaceae, Corynebacterineae, Mycobacteriaceae, Nocardiaceae, Brevibacteriaceae, and Micrococcaceae.

15. The host cell according to claim 13, wherein said host cell is selected from bacteria belonging to the genus Rhodococcus.

16. The host cell according to claim 13, wherein said host cell is the bacterium Rhodococcus erythropolis RG10 as deposited under number DSM 15231 with the DSMZ-Deutsche Sammlung von Mikroorganismen und Zelikulturen.

17. The host cell according to claim 25, which does not contain a functional kstR gene or a homologue or a functional part thereof.

18. A method for producing the heterologous polypeptide in a host cell, comprising transforming the host cell with the recombinant vector of claim 10.

19. (canceled)

20. A method for constitutive expression of a heterologous protein of interest comprising transforming a host cell which does not contain a functional kstR gene or a homoloque or a functional part thereof with a polynucleotide construct wherein the expression of the coding region of said heterologous protein is under control of the kstD promoter.

21. (canceled)

22. A method for identifying compounds that regulate the activity of the kstD promoter comprising exposing a host cell according to claim 14 to at least one compound whose ability to modulate the activity of a kstD promoter is to be determined, and monitoring said cell for modulated kstD promoter activity.

23. The recombinant polynucleotide according to claim 3, further comprising a nucleotide sequence encoding a heterologous polypeptide that is operably linked to the promoter.

24. A vector comprising the recombinant polynucleotide of claim 23.

25. A host cell transformed with the vector of claim 24.

26. The host cell of claim 25, comprising a nucleotide sequence encoding a transcription regulator, wherein the transcription regulator is kstR or a homologue or a functional part thereof.

27. The host cell of claim 26, wherein the transcription regulator comprises SEQ ID NO.: 6.

28. The recombinant polynucleotide according to claim 23, further comprising a nucleotide sequence encoding SEQ ID NO.: 6 or a functional part thereof.

29. A method of inducing expression of a heterologous protein, comprising:
providing a host cell having kstR activity,
transforming the host cell with a vector comprising a nucleotide sequence encoding the heterologous protein operably linked to a kstD promoter from Rhodococcus, and
incubating the transformed host cell in media comprising a concentration of steroid sufficient to lift the repressor function exerted by the kstR activity.


SEQUENCE LISTING (13 sequences)

SEQ ID NO: 1 - DNA, 1543 bp, Rhodococcus erythropolis
  CDS (1)..(1533)
atgcaggactggaccagcgagtgcgacgtgttggtagtcggctccggcggcggagcgctgaccggcgcatataccgccgctgctcagggattgacgacgatcgtcctcgagaaaaccgatcgtttcggcgggacctccgcctactcgggcgcctcgatctggctcccaggtacccaggtgcaggaacgcgccggacttcccgactcgaccgagaatgcccgcacctatctgcgcgcgttgctcggtgacgccgagtccgagcgccaggacgcctacgtcgagaccgctcccgctgtcgtcgctctactcgagcagaacccgaacatcgaattcgagttccgtgcgttccccgactactacaaagccgaaggccggatggacacgggacgctccatcaaccctctcgatctcgatcccgccgacatcggtgacctcgccggcaaggtgcgtccggaactggaccaagaccgcaccggtcaggatcatgctcccggcccgatgatcggtgggcgcgcactgatcggccgtctgctggccgcagttcagagcaccggtaaggcagaacttcgcaccgaatccgtcctcacctccctgatcgtggaagacggccgtgttgtcggcgccgaggtcgaatccggcggcgaaacccagcgaatcaaggcgaaccgcggtgtcctgatggcagcaggcggcatcgaaggcaacgccgagatgcgtgagcaggcaggcacccccggcaaggcgatctggagtatgggtcccttcggcgccaacaccggcgacgcgatctctgccggtattgctgtcggcggcgcaacagccttgctcgatcaggcgtggttctgccccggcgtcgagcagcccgacggcagcgccgccttcatggtcggcgttcgcggtgggctcgtcgtcgacagcgccggtgagcgctacctcaacgagtcgcttccgtacgaccagttcggacgagccatggatgctcacgacgacaacggttctgccgtgccgtcgttcatgatcttcgactcgcgcgagggtggcggactgcccgccatctgcatcccgaacacggcgcccgccaagcacctcgaagccggaacgtgggtcggtgccgacactctcgaagaactcgctgccaagaccggactaccggccgacgcattgcgcagcactgtcgaaaagttcaacgatgccgcaaaactgggcgtcgacgaagagttccatcgcggcgaagacccgtacgacgcgttcttctgcccacccaacggcggtgcgaatgcggcactgacggccatcgagaacggaccgttctacgcggcccgcatcgtcctcagtgacctcggcaccaagggcggattggtcaccgacgtcaacggccgagtcctgcgtgctgacggcagcgccatcgacggcctgtacgccgccggcaacacgagcgcgtcactgagcggccgcttctaccccggccccggagttccactcggcacggctatggtcttctcgtaccgagcagctcaggacatggcgaagtaacgcagttcaa

SEQ ID NO: 2 - protein, 510 aa, Rhodococcus erythropolis
MQDWTSECDVLVVGSGGGALTGAYTAAAQGLTTIVLEKTDRFGGTSAYSGASIWLPGTQVQERAGLPDSTENARTYLRALLGDAESERQDAYVETAPAVVALLEQNPNIEFEFRAFPDYYKAEGRMDTGRSINPLDLDPADIGDLAGKVRPELDQDRTGQDHAPGPMIGGRALIGRLLAAVQSTGKAELRTESVLTSLIVEDGRVVGAEVESGGETQRIKANRGVLMAAGGIEGNAEMREQAGTPGKAIWSMGPFGANTGDAISAGIAVGGATALLDQAWFCPGVEQPDGSAAFMVGVRGGLVVDSAGERYLNESLPYDQFGRAMDAHDDNGSAVPSFMIFDSREGGGLPAICIPNTAPAKHLEAGTWVGADTLEELAAKTGLPADALRSTVEKFNDAAKLGVDEEFHRGEDPYDAFFCPPNGGANAALTAIENGPFYAARIVLSDLGTKGGLVTDVNGRVLRADGSAIDGLYAAGNTSASLSGRFYPGPGVPLGTAMVFSYRAAQDMAK

SEQ ID NO: 3 - DNA, 158 bp, Rhodococcus erythropolis
atcatcgattatgtgtcccggccgcgaacgaccgcgctaattctctcacctggaccacccatctcggcatattgcccgctcagtgggacctggcatggccttccagtgccgtgcggtattccgtggacaccccaccctcttggagtaaggacgcaatg

SEQ ID NO: 4 - DNA, 19 bp, Artificial Sequence
  Description of Artificial Sequenceprimer
ggcgacgttgccgagaatt

SEQ ID NO: 5 - DNA, 624 bp, Rhodococcus erythropolis
  CDS (1)..(624)
atgggggcgacgttgccgagaattgccgaggtcagggacgctgctgagcccagttcggacgagcagcgggcgcgccatgtgcggatgctggaagcggccgccgaattggggaccgagaaagaactctcacgggttcagatgcacgaagttgccaagcgggcaggcgtggccatcggcactctctaccgctatttcccttcgaagacgcacctcttcgtcgctgtgatggtcgagcagatcgatcagatcggcgacagtttcgccaagcatcaggtgcagtcggccaatccgcaggacgccgtgtacgaggtcctggtgcgcgcgactcgcgggttactgcgtcggccggccctttcgactgcgatgctgcagtcgtccagtaccgccaacgtcgcgacggtgccggacgtgggcaagatcgatcgcggcttccggcagatcatcctcgatgcggccgggatcgagaacccgaccgaggaagacaacaccgggttgcgtctgctgatgcagctgtggttcggggtcatccaatcgtgcctcaacggtcgaatttccatcccggatgcggagtacgacatccgcaaggggtgcgacctgcttctggtgaatctctcacgacactga

SEQ ID NO: 6 - protein, 207 aa, Rhodococcus erythropolis
MGATLPRIAEVRDAAEPSSDEQRARHVRMLEAAAELGTEKELSRVQMHEVAKRAGVAIGTLYRYFPSKTHLFVAVMVEQIDQIGDSFAKHQVQSANPQDAVYEVLVRATRGLLRRPALSTAMLQSSSTANVATVPDVGKIDRGFRQIILDAAGIENPTEEDNTGLRLLMQLWFGVIQSCLNGRISIPDAEYDIRKGCDLLLVNLSRH

SEQ ID NO: 7 - DNA, 20 bp, Artificial Sequence
  Description of Artificial Sequenceprimer
tcagtgtcgtgagagattca

SEQ ID NO: 8 - DNA, 35 bp, Artificial Sequence
  Description of Artificial Sequenceprimer
ataaagcttatcgattatgtgtcccggccgcgaac

SEQ ID NO: 9 - DNA, 35 bp, Artificial Sequence
  Description of Artificial Sequenceprimer
ataggtaccatatgtgcgtccttactccaagaggg

SEQ ID NO: 10 - DNA, 27 bp, Artificial Sequence
  Description of Artificial Sequenceprimer
gcgcatatggctaagaatcaggcaccc

SEQ ID NO: 11 - DNA, 30 bp, Artificial Sequence
  Description of Artificial Sequenceprimer
gcgggatccctacttctctgctgcgtgatg

SEQ ID NO: 12 - DNA, 30 bp, Artificial Sequence
  Description of Artificial Sequenceprimer
ggccatatgttgaccacagacgtgacgacc

SEQ ID NO: 13 - DNA, 30 bp, Artificial Sequence
  Description of Artificial Sequenceprimer
gccactagttcactgcgctgctcctgcacg